(19) United States
(12) Patent Application Publication
(10) Pub. No.: US 2004/0196998 A1
(43) Pub. Date: Oct. 7, 2004

(54) Extra-ear hearing

(76) Inventor: Paul Noble, Salt Lake City, UT (US)

Correspondence Address: Lynn G. Foster, 602 East 300 South, Salt Lake City, UT, 84102, US

(21) Appl. No.: 10/407,739

(22) Filed: Apr. 4, 2003

(51) Int. Cl. H04R 25/00; A61N 1/00; A61N 1/04; A61N 1/05; A61N 1/06

(52) U.S. Cl. 381/331; 600/25; 607/137

(57) ABSTRACT

Novel non-invasive tactile hearing apparatus and methodology are disclosed which apparatus comprises tactile transmitter contiguously in contact with the skin of the user and, utilizing nerves as opposed to ears, enhances the hearing of those who are hearing-impaired and provides hearing to those who are legally deaf.

### FIELD OF THE INVENTION

This invention relates to human hearing and, more particularly, to artificial extra-ear non-invasive hearing apparatus and methods for those who are either hearing impaired or deaf.

### BACKGROUND

Apparatus and methodology for enhancing hearing, short of surgery, has in the past comprised hearing aids, which assist the ears to enjoy improved hearing. Hearing aids of the past may be classified as follows:

1. The common amplified non-invasive hearing aid, which is inserted into the hearing canal and simply amplifies sound. This type of device cannot differentiate different levels of foreground and background noise and, therefore, amplifies all sound equally, thus making it difficult for the user to distinguish mixed sound levels and clarity of speech such as occurs in a crowded place.

2. The newer, more expensive non-invasive types of hearing aids which have circuits for speech clarity and noise reduction and are also inserted into the hearing canal.

They still have some drawbacks as to clarity of sound and some cutoff sounds above and below certain levels.

3. The third type of hearing aid is invasive and, therefore, requires implant surgery. Wires are surgically implanted into the cochlear nerves and an interface is located behind the ear where a standard hearing device makes an interface connection. This type of device works in conjunction with the ear for those who have mechanical damage. While this type of device works, it still has problems in normal speech recognition.

None of the devices mentioned immediately above works on a person who is born with or through disease lacks nerves connecting from the ears to the brain.

Current hearing devices falling within the three categories mentioned above, although improved, still have limitations. Typically, they are bulky or have to be inserted into the ear, the partial exposure of which may be embarrassing to some.

Availability of non-invasive tactile hearing apparatus and methodology utilizing nerves, not ears, to enhance hearing of those partially impaired or deaf, embodying the present invention, is a major step forward.

### BRIEF SUMMARY AND OBJECTS OF THE PRESENT INVENTION

In brief summary, the present invention overcomes or substantially alleviates prior problems in the field of assisted human hearing, as such pertains to the hearing-impaired and those who are legally deaf. The present invention provides novel non-invasive tactile hearing apparatus and methodology, which contiguously contacts the skin of the user and, utilizing nerves as opposed to ears, enhances the hearing of those who are hearing-impaired and provides hearing to those who are legally deaf.

With the foregoing in mind, it is a primary object of the present invention to overcome or substantially alleviate prior problems pertaining to assisted human hearing for the hearing-impaired and/or those who are legally deaf.

It is an object of paramount importance to provide novel non-invasive tactile hearing apparatus and methodology to enhance the hearing of those who are hearing-impaired and/or provide hearing to those who are deaf.

Another valuable object is the provision of novel tactile non-invasive hearing apparatus and methodology by which signals of predetermined characteristics are delivered not to the ears, but to the skin and thence along nerves to a brain location where the transmitted signal is interpreted as being sound.

These and other objects and features of the present invention will be apparent from the detailed description taken with reference to accompanying drawings.

### BRIEF DESCRIPTION OF THE DRAWINGS

FIG. 1 is a block diagram of one form of the present invention depicting component and the signal path; and

FIG. 2 is a diagram showing the relationship between the circuit diagrams of FIGS. 2A and 2B; and

FIGS. 2A and 2B, taken together comprises a circuit diagram of the embodiment of FIG. 1.

### DETAILED DESCRIPTION OF THE ILLUSTRATED EMBODIMENTS

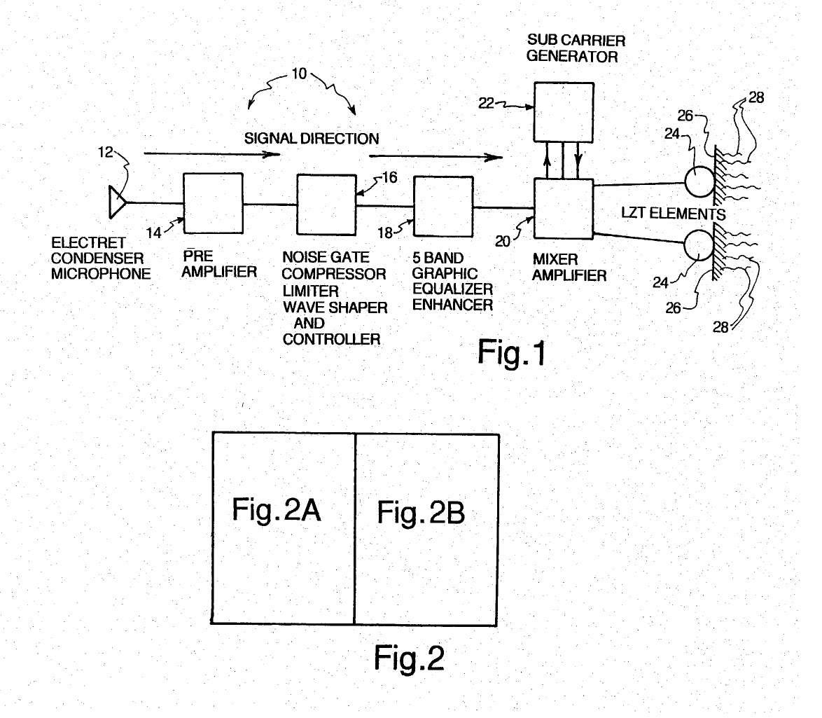

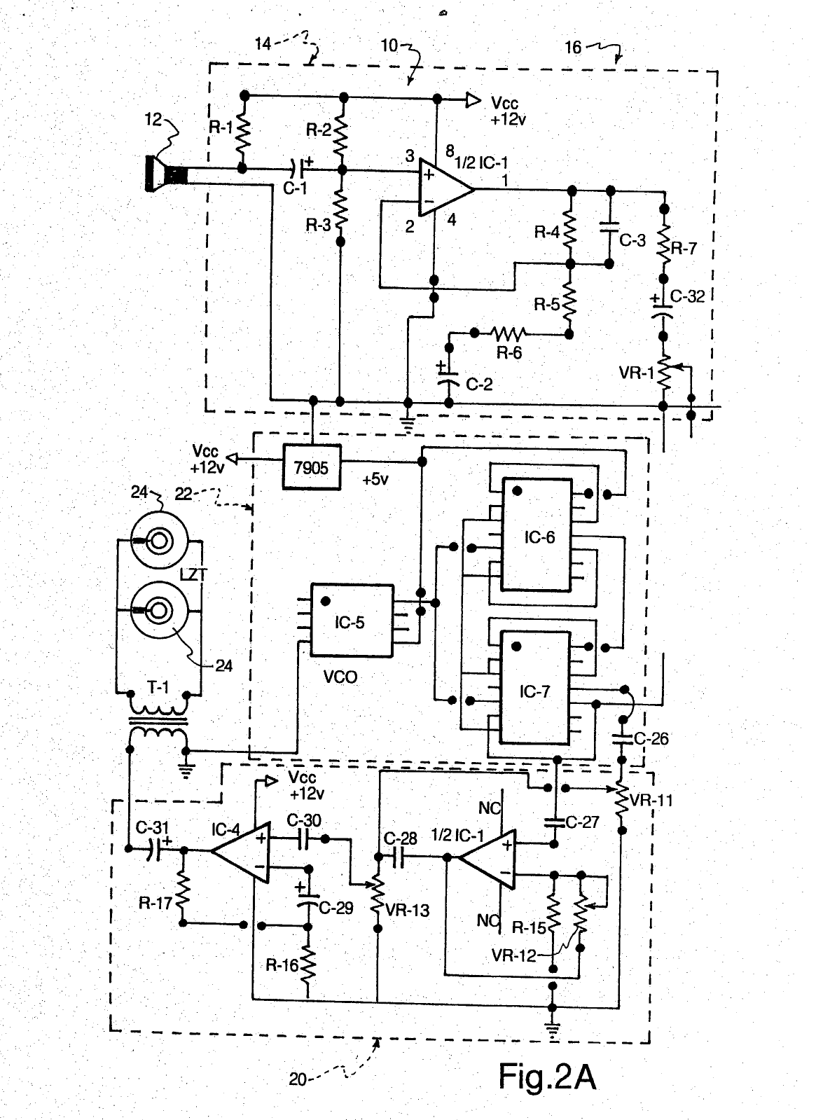

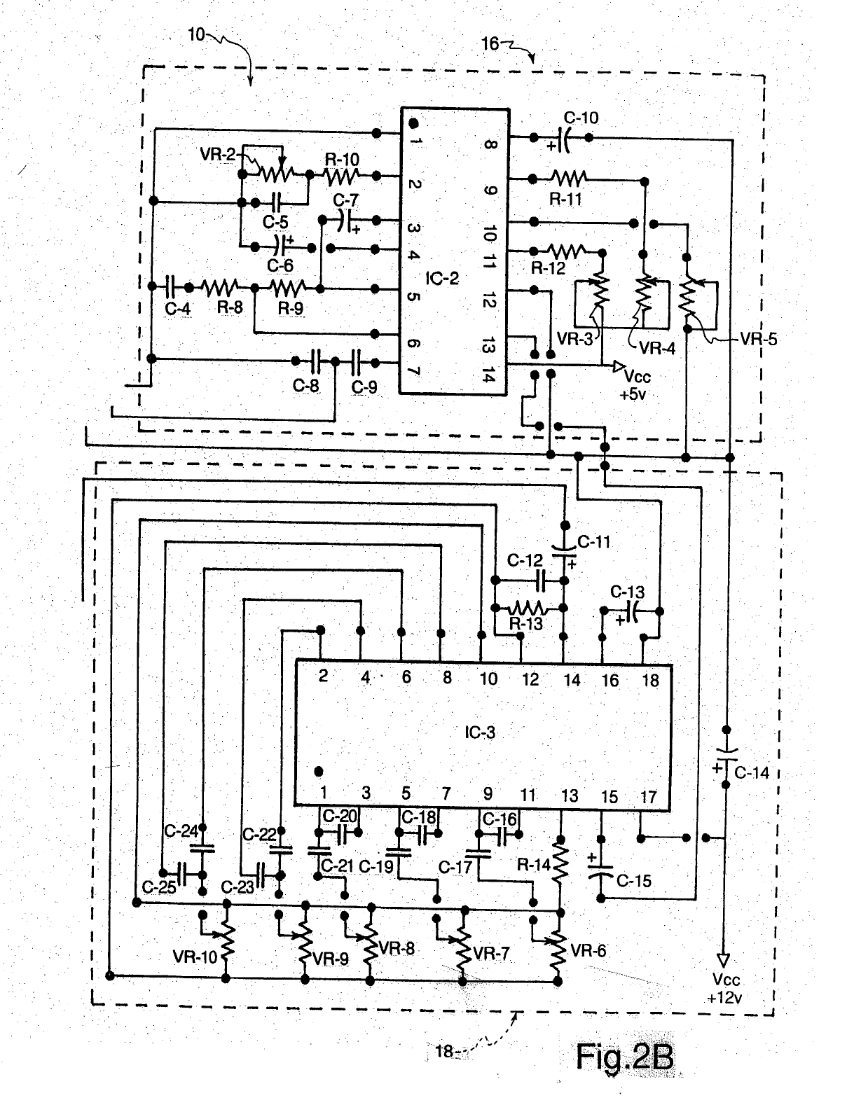

Reference is now made to the drawings wherein like numerals are used throughout to designate like parts. FIGS. 1 and 2 illustrate one physical hearing embodiment implementing the principles of the present invention. This hearing embodiment is generally designated 10 in FIGS. 1 and 2.

With reference specifically to FIG. 1, sound enters the apparatus 10 through a full frequency electret condenser microphone 12 and is amplified at pre-amplifier 14 to a desired level. One desired level may be −20 db. The signal emanating from pre-amplifier 14 is communicated through a control-filter device, (aka noise gate/compressor/limiter, wave shaper and controller), generally designated 16. The signal emanating from control-filter device 16 is passed through a 5-band graphic equalizer and enhancer, generally designated 18. The signal emanating from equalizer and enhancer 18 is communicated to a mixer amplifier, generally designated 20, which also receives a carrier signal from sub-carrier generator 22. The two signals are superimposed at mixer amplifier 20 sot that the modulated signal is amplified to a suitable drive level. One suitable drive level may be 50-60 volts rms. The output from the mixer amplifier 20 is communicated to two spaced LZT transmitter elements 24 and 24 and thence through the skin 26, contiguous with the element 24, to adjacent nerve ends 28 and thence along a major nerve, such as the trigeminal nerve, using vascular conduction and transneural induction to the location in the brain where the modulated signal is interpreted as being sound.

With reference to FIG. 2, sound enters the apparatus 10 at electret condenser microphone 12 and is brought up to a line level signal, e.g., −20 db by ½ of integrated circuit IC-1 (which may be TL072) pin 3. The signal passes through resistor R-7, which may be a 100 ohm resistor, and thence through capacitor C-32, which may be a 2.2 uf electrolytic capacitor.

The gain level is set to the next stage by variable resistor VR-1, which may be 10 k ohm trim pot. The signal then passes through capacitor C-9, which may be a 0.01 uf capacitor, and into pin 7 of integrated circuit IC-2, which may be a SSM2166. This is the noise gate input (used to set the background level noise cut-off threshold). The threshold is set by variable resistor VR-2, which may be a 10 k ohm trim pot.

The signal then passes through a compressor stage within integrated circuit IC-2. This keeps unwanted high volume peaks from over-driving the circuit, which could cause distortion. This threshold is set by variable resistor VR-3, which may be a 50k ohm trim pot.

The signal then passes through an internal limiter, which limits the maximum volume output level. This keeps the output level smooth so as not to clip the signal issued to the next device and cause unwanted harmonic overtones misinterpreted by the brain.

The signal then flows out of pin 13 of integrated circuit IC-2, the level of which is controlled by variable resistor VR-5 (which may be a 25 k ohm trim pot), to integrated circuit IC-3 at pin 15 through capacitor C-15, (which may be a 10 uf electrolytic capacitor). Integrated circuit IC-3 may be a BA3812L, which is a 5 band graphic equalizer used to set the enhancement frequency for a particular user. The individual frequencies are controlled by five resistor-capacitor (R-C) networks, which are described below.

A low frequency range, such as 200 Hz, is controlled by the network combination of variable resistor VR-10, which may be a 100 k ohm trim pot, capacitor C-24, which may be a 0.027 uf capacitor and capacitor C-25, which may be a 1 uf capacitor.

A low to mid range, such as 500 Hz, is controlled by the network combination comprising variable resistor VR-9, which may be a 100 k-ohm trim pot, capacitor C-22, which may be a 0.0032 uf capacitor and capacitor C-23, which may be a 0.33 uf capacitor.

A mid-range frequency, such as 1000 Hz, is controlled by the network combination comprising variable resistor VR-8, which may be a 100 k ohm trim pot, capacitor C-20, which may be a 0.0027 pf capacitor and capacitor C-21, which may be a 1 uf capacitor.

A mid to high frequency, such as 3000 Hz, is controlled by the network combination comprising variable resistor VR-7, which may be a 100 k-ohm trim pot., capacitor C-18, which may be a 820 pf capacitor, and capacitor C-19, which may be a 0.01 uf capacitor.

A high frequency, such as 10,000 Hz is controlled by the network combination comprising variable resistor VR-6, which may be a 100 k-ohm trim pot, capacitor C-16, which may be 270 pf capacitor, and capacitor C-17, which may be a 0.01 uf capacitor.

The signal then flows out of pin 14 of integrated circuit IC-3 through capacitor C-11, which may be a 10 uf electrolytic capacitor, to inputs of both integrated circuit IC-6 and IC-7, where a pulse width and IC-modulation signal is triggered by the incoming audio signal. Some of the audio signal is diverted through capacitor C-27, which may be a 0.01 uf capacitor, to the input pin 2 of the second half of integrated circuit IC-1 (which may be a TL0-72 or a 5532).

A square signal, which may be 50 kHz, is produced by integrated circuit IC-5 (994M) at pin 8 and is simultaneously fed to both integrated circuit IC-6 and integrated circuit IC-7 at pin 5. Two half adders integrated circuit IC-6 and integrated circuit IC-7 cancel this signal to “0” output when no audio is present at the inputs thereof. This means that no burst frequency is present riding on the audio carrier. When audio is applied to the inputs of integrated circuits IC-6 and IC-7, a varying amplitude/frequency modulated pulse width carrier wave is added to the original audio signal through capacitor C-26, which may be a 0.01 uf capacitor, and variable resistor VR-11, which may be a 100 k ohm trim pot.

This signal is mixed at the junction of capacitor C-28, which may be a 1 uf capacitor, and variable resistor VR-13, which may be a 100 k-ohm trim pot. This mixed signal is fed through capacitor C-30 with the audio from the second half of integrated circuits IC-1 (which may be a TL072 or a 5532) mixed. This signal enters integrated circuit IC-4 at pin 2, where it is raised to a suitable drive level, which may be 12 v rms. This signal passes through capacitor C-31, which may be a 1000 uf electrolytic capacitor, into the primary of transformer T1. Transformer T1 may be a specially wound toroidal step up audio transformer, which may have a ratio of 1:4. The output, which may be 50 kHz 60 v rms, is produced to drive the LZT 24 (which may be lead zirconium titanate elements), placed against the skin, perhaps just in front of the ears, and there secured in position by means of a headphone-like band, for example.

In one of many possible configurations, sound enters through an electret condenser microphone 12 having a frequency response of 20-20 khz (20 cycles to 20,000 cps). See FIG. 1. The sound is amplified through a preamplifier 14. The output of the preamplifier 14 then goes through compressor/limiter/noise gate 16, which adds clarity by removing unwanted background noise thus increasing speech recognition. The sound then passes through graphic equalizer 18, which either cuts or enhances certain frequencies (adjusted for each particular user). From the equalizer 18 it passes through the mixer amplifier 20. The signal is applied to both the input stage of the 741 op/amp and the PCM devices (7490 s). An ultrasonic frequency in the range of 46-55 khz provided by the 994 device 22 is modulated and mixed with the PCM by the two 7490 devices. The PCM signal is mixed with the audio signal of the 741 op/amp. This signal is amplified again by the LM 383 device and fed to the 1:4 ratio transformer (T1) used to create the 60-70 volts necessary to drive the LZT devices 22 and 24.

Resistor R-1 controls the voltage of the electricity communicated to the condenser microphone. Resistors R-2 and R-3 function to bias integrated circuit IC-1. Capacitor C-1 is a coupling capacitor. Collectively, resistor R-4 and capacitor C-3 comprise a feedback circuit for gain on integrated circuit IC-1. Resistors R-5 and R-6 together with capacitor C-2 comprise a circuit which sets the frequency limit on integrated circuit IC-1.

Capacitors C-10 and C-6 comprise DC filter capacitors for noise stability. Resistors R-10, R-11 and R-12 are current limiting resistors. Capacitor C-5 comprises a RF trap (filter), while capacitor C-7 provides frequency stability. At capacitor C-4 and resistors R-8 and R-9 comprise a gain limiting circuit. Capacitor C-8 comprises an RF trap (filter). Capacitors C-13 and C-14 comprise DC filter capacitors for noise stability. Capacitor C-12 and resistor R-13 collectively comprise a gain limiting circuit. Resistor R-14 functions as a filter, while variable resistor VR-4 limits gain.

Element 7905 comprises a five volt DC regulator.

Collectively resistors R-17, R-16 and capacitor C-29 comprise a feedback circuit for gain for integrated circuit IC-4. Variable resistor VR-12 and resistor R-15 provide for pre-gain adjustment for one-half of integrated circuit IC-1.

Not by way of limitation, but as exemplary, the following components and values may be used:

All resistors may be ¼ 1%.

| | | |
|---|---|---|
| R-1. 8 & 9 | 10 | k ohm |
| R-2, R-3 & R-6 | 27 | k ohm |
| R-4 | 33 | k ohm |
| R-7 | 100 | ohm |
| R-5 | 1.5 | k ohm |
| R-10, 11 & 12 | 1 | k ohm |
| R-13 | 68 | k ohm |
| R-14 | 6.8 | k ohm |
| R-15 | 10 | ohm |
| R-16 | 2.2 | ohm |
| R-17 | 220 | ohm |

All capacitors may be 1% Polypropylene or Polycarbonate
16 VDC except electrolytics which are Aluminum/Mylar 16 VDC.

| | | |
|---|---|---|
| C-3 | 2 | pf |
| C-4, 21, 25 & 28 | 1 | uf |
| C-8 | 33 | pf |
| C-9, 17 & 27 | .01 | uf |
| C-12 | 1000 | pf |
| C-16 | 270 | pf |
| C-18 | 820 | pf |
| C-19 | .033 | uf |
| C-20 | .0027 | uf |
| C-22 | .0032 | uf |
| C-23 | .33 | uf |
| C-24 | .027 | uf |

Electrolytics may be:

| | | |
|---|---|---|
| C-1 & 32 | 2.2 | uf |
| C-2, 6, 7, 11 & 15 | 10 | uf |
| C-10 | 4.7 | uf |
| C-13 & 14 | 100 | uf |
| C-29 | 470 | uf |
| C-31 | 1000 | uf |

Trim Pots may be Centralab Micro miniature
Film Type PC Mount 5%.

| | | |
|---|---|---|
| VR-1, 2 & 11 | 10 | k ohm |
| VR-3 | 50 | k ohm |
| VR-4 & 12 | 1 | Meg-ohm |
| VR-5 | 25 | k ohm |
| VR-6, 7, 8, 9, 10 & 13 | 100 | k ohm |

Integrated circuits may be current versions.

| | |
|---|---|
| IC-1 | TO072 or 5532 |
| IC-2 | SSM2166 |
| IC-3 | BA3812L |
| IC-4 | LM383 |
| IC-5 | 994M |
| IC-6 & 7 | 7490 |
| T1 | 5:1 Ratio 1A Audio former |
| LZT | .75″ Lead Zirconium Titanate |
| | Ultra Sonic Drivers |
| 12 | 20-20 khz. ⅜″ dia. |
| | 1.5 V Electret Condenser Microphone |

While, the theory by which the present invention operates is not believed to be fully understood, the following explains the present view.

Contrary to popular belief, it has been found that humans can hear frequencies above 20 kHz, if a transducing element is coupled directly to the skin of the head, neck, upper torso or elsewhere. The head is presently preferred. Ultrasonic hearing is a basal cochlear transduction process. Ultrasonic stimuli are audible to submerged mammals, including humans, however the same stimuli presented in air are generally inaudible. Seals have adapted to life in and out of the water. Seal upper hearing limit by underwater bone conduction (bc) is approximately 50 kHz higher than the upper limit of hearing by air conduction (ac). It is believed, the same relation holds true in man in which the upper limit of (bc) hearing is more than two octaves higher than the 20 kHz ac limit. Human (bc) thresholds increase by 50 dB (decibels) per octave between 10 and 20 kHz and only 15 (dB) per octave above 20 kHz.

Seals in contrast show little bc loss between 10 and 20 kHz, but have a 50 dB per octave increase in thresholds between 32 and 64 kHz and only a 15 dB or so difference above 90 kHz. Thus the pattern of bc underwater in seals and humans is similar with the seal being more sensitive to ultrasonic energy. Thresholds of the California sea lion for tones “ultrasonic to it” above the limit are within the same ultrasonic intensity range found in normal hearing humans. Seals show a number of anatomical adaptations for bc hearing including enlarged basal cochlear whorl, enlarged round window and enlarged cochlear aqueduct. The same structures are hypothesized to play essential roles in human ultrasonic hearing. Psychophysical tests and modeling suggest that ultrasound propagates through bodily fluid and enters the ear via fluid channels such as the aqueduct. The tip of the basilar membrane is hypothesized to be the resonant structure that moves in response to ultrasonic stimulation.

Frequency discrimination is present in the transition zone between the upper limit of ac hearing and ultrasonic bc hearing, allowing the possibility for limited function such as echolocation of objects in space under conditions of limited light or blindness and ultrasonic speech perception.

The cochlear aqueduct connects the cerebral spinal fluid to the perilymphatic fluids of the middle ear, these fluids in turn surround the scala media which contains the endolymphatic fluids and proceeds to the tip of the cochlea. This would explain why transneural induction works so well when one of the skin transmitters is placed along the spine.

The saccule is not an organ of hearing and, while it does contain sensitive hair receptors, they have not been shown to act in any way that is connected to the nerves associated with hearing.

The cochlea acts as a physical filter to the traveling pressure wave entering it through the endolymphatic fluid. This wave is usually initiated by the action of the tympanic membrane, however there is no reason why this should be the only vehicle of transmission. Dolphins and other toothed whales, which have extra ordinal hearing in the upper ranges, have a channel filled with a fatty substance in their lower jaw that connects to their tympanoperiotic bone and then to the cochlear duct. Once in the cochlear duct, the pressure wave travels down the basicular membrane. The stiffness of the basicular membrane varies evenly with the length thereof and any one point will resonate at a particular frequency along the cochlear membrane. This resonate motion is sensed by hairs which in turn stimulate the neurons which communicate with the brain.

The Batteau beat frequency test not withstanding, no reason was seen to not consider vascular conduction as a viable model for transneural induction. As an added reason for accepting the vascular conduction model, it has recently been shown that dolphins can hear with their flippers and skin over most of their body nearly as well as with the organs usually associated with hearing.

It has been believed that all hearing was by basal cochlear transduction. The skin is the largest organ on the body consisting of trillions of nerve endings. These nerves can be stimulated in such a way so they will transmit information directly to the brain.

The present invention is a powerful brain-based, non-invasive apparatus and methodology. It not only offers hearing enhancement but, if alpha or theta signals are fed into it, the brain can be moved into any state desired. The mind can recognize up to 600 words per minute, but rarely do you hear more than 300. Because the present invention imprints directly on the cerebral cortex, the information goes directly to the long term learning centers of the brain. This open up tremendous new avenues for education as well.

The invention may be embodied in other specific forms without departing from the spirit or essential characteristics thereof. The present-embodiments are therefore to be considered in all respects as illustrative and not restrictive, the scope of the invention being indicated by the appended claims rather than by the foregoing description, and all changes which come within the meaning and range of equivalency of the claims are therefore intended to be embraced therein.

What is claimed and desired to be secured by Letters Patent is:

1. A sensory non-invasive method by which extra-ear hearing is accommodated, comprising the acts of:
placing at least one tactile transmitter contiguous with the skin;
transmitting a audio signal of predetermined characteristics to nerve ends in the immediate vicinity of the skin at the placing site and thence along a nerve to the brain at a location where the signal is interpreted as being sound.

2. A non-invasive method according to claim 1 wherein the placing act is at the skin of the face of a user.

3. A non-invasive method according to claim 1 wherein the placing act is at a skin location other than the face.

4. A method according to claim 1 wherein the transmitting act comprises communicating the signal along the nerve.

5. A method according to claim 1 wherein the signal is intelligible to the brain.

6. A non-invasive method according to claim 1 wherein the signal of the tactile transmitter is prepared by equalizing, compressing and clarifying a sound signal.

7. A non-invasive method according to claim 6 wherein the equalizing compressing clarifying sound of and limits the amplification of the sound signal.

8. A non-invasive method according to claim 6 wherein the sound signal is passed through a noise gate to alleviate background noise.

9. A non-invasive method according to claim 6 wherein the sound signal is mixed with a carrier signal, fed through a piezo electric transducer, before delivery to the tactile transmitter.

10. A non-invasive method according to claim 1 wherein the transmitting step comprises vascular conduction and transneural induction.

11. A non-invasive method according to claim 1 wherein the characteristics of the transmitted signal obtained through use of circuitry by which the signal is amplified and clarified.

12. A non-invasive method according to claim 1 wherein the transmitted signal comprises a sound signal superimposed upon a carrier signal.

13. A non-invasive method according to claim 12 wherein the carrier signal comprises a pulsed time variant ultrasonic frequency whereby, when combined with the sound signal, creates neural firing from the tactile transmitter to nerve ends adjacent to the skin.

14. A non-invasive apparatus by which extra-ear hearing is accommodated comprising:
a tactile transmitter for contiguous placement on the skin;
a signal generator by which a signal of predetermined characteristic is formulated;
a circuit by which the generated signal is communicated through the tactile transmitter to nerve ends at the skin and thence to the brain at a location where the signal is received-as sound.
